(19) United States
(12) Patent Application Publication
(10) Pub. No.: US 2010/0009012 A1
(43) Pub. Date: Jan. 14, 2010

(54) CHEWABLE PHARYNGEAL INFLAMMATION SYMPTOM RELIEF COMPOSITION

(76) Inventor: N. David Zukkoor, Farmington Hills, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/501,639

(22) Filed: Jul. 13, 2009

Related U.S. Application Data
(60) Provisional application No. 61/080,013, filed on Jul. 11, 2008.

(51) Int. Cl. A61K 33/30

(52) U.S. Cl. 424/643

(57) ABSTRACT

A chewable composition for treating pharyngeal inflammation is provided that includes a first chewable prostaglandin inhibitor tablet that includes a prostaglandin inhibitor along with those excipients needed to form a compressed chewable tablet. The first chewable tablet is devoid of ascorbic acid, citric acid, and zinc cations. A second chewable supplement tablet of the composition includes an immune system activating organic acid of ascorbic acid, citric acid, or a combination thereof. The second chewable supplement tablet also includes zinc cations. The components of the second chewable supplement tablet are readily segregated into multiple tablets, with each of the multiple tablets containing similar or dissimilar active ingredient constituents and concentrations. A process for treating infective throat inflammation is provided that includes a subject simultaneously or successively chewing the inventive composition so as to adhere tablet particulate containing the prostaglandin inhibitor and the other components to the inflamed tissue associated with infection.

### CROSS-REFERENCE TO RELATED APPLICATION

This application claims priority of U.S. Provisional Patent Application Ser. No. 61/080,013 filed Jul. 11, 2008, the contents of which are incorporated herein by reference.

### FIELD OF THE INVENTION

The present invention in general relates to a composition for symptom treatment associated with inflammation of the throat and in particular to a chewable composition providing near term symptom relief and agents active to reduce inflammation severity and duration.

### BACKGROUND OF THE INVENTION

The development of pharyngeal inflammation is associated with a variety of viral and bacterial infections. Standard practice is generally not to treat the symptom of a sore throat until the underlying infection has progressed to an extent indicative of a potentially severe or contagious infection such as that, for example, associated with beta-hemolytic streptococcus. A problem associated with the current therapeutic approach is that other than providing palliative lozenges to temporarily suppress throat discomfort, conventional remedies for unspecified throat discomfort routinely fail to invoke multiple modes of action to shorten the duration and severity of the underlying source of the inflammation.

Prostaglandin inhibitors such as acetylsalicylic acid are particularly effective at reducing inflammation, yet systemic administration of prostaglandin inhibitors is not especially effective owing to the lag time in gastric absorption and the comparatively low concentration at the site of inflammation through systemic administration. Liquid forms of orally administered prostaglandin inhibitors, while effective in topically coating inflamed throat tissue, tend to be cleared too quickly by saliva to achieve the desired therapeutic result.

Thus, there exists a need for an oral tablet capable of providing multiple mode treatment of nonspecific throat inflammation.

### SUMMARY OF THE INVENTION

A chewable composition for treating pharyngeal inflammation is provided that includes a first chewable prostaglandin inhibitor tablet that includes a prostaglandin inhibitor along with those excipients needed to form a compressed chewable tablet. The first chewable tablet is devoid of all but impurity levels of ascorbic acid, citric acid, and zinc cations. A second chewable supplement tablet of the composition includes an immune system activating organic acid of ascorbic acid, citric acid, or a combination thereof. The second chewable supplement tablet also includes zinc cations. The components of the second chewable supplement tablet are readily segregated into multiple tablets, with each of the multiple tablets containing similar or dissimilar active ingredient constituents and concentrations. A specific chewable composition includes between 50 and 800 milligrams of acetylsalicylic acid in a first tablet and the remaining components of ascorbic acid present between 50 and 500 milligrams, zinc cations present from between 0.001 and 0.4 milligrams, along with pharmaceutically acceptable excipients compressive to form chewable tablet forms with the ascorbic acid and zinc cations being present in a single tablet or segregated into separate tablets. The composition optionally includes a natural essential oil and/or capsaicin.

A process for treating infective throat inflammation is provided that includes a subject simultaneously or successively chewing the inventive composition so as to adhere tablet particulate containing tie prostaglandin inhibitor and the other components to the inflamed tissue associated with infection.

### DETAILED DESCRIPTION OF THE PREFERRED EMBODIMENTS

The present invention has utility as a composition for the treatment of throat inflammation. The present invention includes a number of ingredients having different modes of action in the treatment of throat inflammation. The inventive composition is delivered in a form providing contact absorption of a prostaglandin inhibitor directly to inflamed pharyngeal tissue, and discomfort is rapidly suppressed.

An inventive composition includes a prostaglandin inhibitor illustratively including willow bark extracts, such as white willow bark; acetylsalicylic acid; indomethacin; acetaminophen; ibuprofen; or pharmaceutically acceptable salts of the aforementioned. An effective dose of prostaglandin inhibitor intended to adhere to pharyngeal tissue depends on factors illustratively including the identity of the prostaglandin inhibitor, solubility, die prostaglandin inhibitor in saliva, and granule size. Typical prostaglandin inhibitor dosage is between 100 and 600 milligrams. In the exemplary instance where the prostaglandin inhibitor is acetylsalicylic acid, the dosage is between 200 and 500 milligrams and preferably between 300 and 400 milligrams. Optionally, the prostaglandin inhibitor includes two or more of the aforementioned prostaglandin inhibitors acting in concert.

An inventive composition also includes a quantity of immune system active organic acid. Immune system active organic acids operative herein include ascorbic acid and citric acid, combinations thereof, and salts thereof. The mechanism by which ascorbic acid operates to lessen the severity and duration of infection through bolstering of the immune system is well known to the art. Frei, B., Stocker, R. & Ames, B. N. Ascorbate is an outstanding antioxidant in human blood plasma. Proceedings of the National Academy of Sciences, USA. 1989, 86, 6377-6381.

A preferred embodiment of an inventive composition includes a first tablet that is a conventional chewable tablet containing only the prostaglandin inhibitor and necessary adjuvants conventional to the art, and a second chewable tablet inclusive of the other inventive composition active agents. Usage entails the simultaneous chewing of the first tablet and second tablet to achieve therapeutic or prophylactic effect. It is appreciated that the health food supplement and/or natural product component tablet components are readily further subdivided such that these components are present in two or more non-prostaglandin inhibitor containing tablets. It has been discovered that the storage stability of the inventive composition is enhanced through segregating aspirin or other exemplary prostaglandin inhibitors separate from the natural product components.

An inventive composition typically includes between 50 and 500 milligrams of immune system activating organic acid and preferably between 100 and 300 milligrams per tablet.

An inventive composition also includes a quantity of divalent zinc ions. Zinc ions are readily delivered as an inorganic salt, or as a complex formed with a ligand illustratively including ethylenediaminetetraacetic acid (EDTA), diethylenetriaminepentaacetic acid (DTPA), nitrilotriacetic acid (NTA), iminodiacetic acid (IDA), iminotriacetic acid (ITA), ethylenediamine (En), N,N′-diethylenediamine (Den), diethylenetriamine (DTN), diethylenetetramine (Trien), triaminotriethylene amine, propylenediamine, glycolic acid, hydroxybutyric acid, salicylic acid, benzoic acid, ascorbic acid, citric acid, or combinations thereof. Preferably, the zinc ions are present as a water-soluble salt having a counter ion illustratively including chloride, bromide, nitrate, acetate, and propionate. Preferably, zinc ions are delivered as a water-soluble salt. Without intending to be bound by a particular theory, complexed zinc ions are less efficient in entering inflamed throat tissue cells through ion channel pumps when such ions are present in chelated form. It is appreciated that tablet formulation with powders of zinc ion salt and immune system activating acid readily occurs without chelation therebetween through moisture control. According to the present invention, zinc ions are present in a quantity of from 0.001 to 0.1 milligrams with the weight quantity of zinc ions calculated independent of the mass of counter ion or ligands. Preferably, zinc ions are provided in an inventive composition in an amount of from 0.005 to 0.05 and most preferably from 0.01 to 0.02 milligrams.

An inventive composition also optionally includes as an active ingredient a quantity of capsaicin. It is appreciated that the capsaicin need not be present at all, but if present can be in purified form, as well as with a quantity of plant material in which capsaicin is naturally occurring. Naturally occurring plant products containing capsaicin illustratively include seeds and/or fruit of pepper plants of the genus Capsicum and include varieties such as Capsicum annuum which includes many common varieties such as bell peppers, paprika, jalapeños, and the chiltepin Capsicum frutescens, which includes the cayenne and tabasco peppers; Capsicum chinense, which includes the naga, habanero and Scotch bonnet; Capsicum pubescens, which includes the rocoto peppers; and Capsicum baccatum, which includes the aji peppers. It is appreciated that depending on the nature of the plant material, capsaicin makes up anywhere from 0.01 to 5 percent of the weight of the plant material. Capsaicin present in quantities as low as 0.00001 milligrams is effective in inducing pharyngeal tissue dilation that facilitates absorption of other active components inclusive of the prostaglandin inhibitor, organic acid, and zinc ions. In the instance where the capsaicin is present in cayenne pepper powder, quantities on the order of 0.0001 to 0.008 milligrams stimulate active component contact absorption by inflamed pharyngeal tissue without creating an intolerable or unpleasant sensation of spice induced mouth burning.

Additional active ingredients optionally included within an inventive composition include a quantity of a natural essential oil having biocidal properties. A natural essential oil is present in an amount dependent upon the biocidal properties of the specific natural essential oil. These properties are known to vary between different oils and individual plants. Antibacterial agents that are useful in the present invention are the so-called “natural” antimicrobial actives, referred to synonymously herein as natural essential oils. These actives derive their names from their natural occurrence in plants. Natural essential oil antimicrobial actives according to the present invention include oils of anise, lemon, orange, oregano, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, gum benzoin, basil, fennel, fir, balsam, menthol, ocmea origanuna, Hydrastis carradensis, Berberidaceae daceae, Ratanhiae and Curcuma longa. Also included in this class of natural essential oils are the key chemical components of the plant oils that have been found to provide the antimicrobial benefit. These chemicals include, but are not limited to, anethol, catechol, camphene, carvacrol, eugenol, eucalyptol, ferulic acid, farnesol, hinokitiol, tropolone, limonene, menthol, methyl salicylate, thymol, terpinol, verbenone, berberine, ratanhiae extract, caryophellene oxide, citronellic acid, curcumin, nerolidol and geraniol. A natural essential oil is active in quantities from 0.01 to about 25 weight percent. Preferably, a natural oil is present from 0.1 to 3 total weight percent. More preferably, a natural oil is present from 0.3 to 1 total weight percent.

As used herein, the term “antimicrobial activity” is defined to include an inhibition zone of at least 2 millimeters around a colony of pathogenic or potentially pathogenic skin colonizing gram-positive, gram-negative or fungal organisms on an agar plate where a test substance is applied to a planar colony at a concentration of 0.5 microliters per square centimeter of organism growth medium surface area, following 24 hours of incubation.

In a preferred embodiment, the natural essential oil is the complex mixture of substances making up oregano oil. While it is appreciated that this and other natural oils vary in the amount and ratio of constituent components illustratively based on plant variety, growing conditions, plant portions harvested for extraction, and extraction process, these natural oils are nonetheless operative with varying amounts of such natural oils being needed as an active ingredient based on antimicrobial activity. Typically, a natural essential oil is present from 0 to 100 milligrams. The inferred ranges for specific natural essential oils are provided below, in the instance where an inventive composition in fact includes an optional natural essential oil.

In addition to the active ingredients of a prostaglandin inhibitor, an organic acid, zinc ions and optional active agents of silver, capsaicin, natural essential oil, or a combination thereof, an inventive tablet composition includes excipients present to facilitate the formation of compressible chewable tablets. The water-disintegratable, compressible carbohydrate used in the present invention includes carbohydrate materials conventionally used in tablets. The carbohydrates facilitate the breakup of the dosage form after oral administration, and are described in Lieberman et al., Pharmaceutical Dosage Forms, Marcel Dekker, Inc., New York, 2 Ed. Vol. 1, pp. 205-209 (1990). Representative disintegratable, compressible carbohydrates illustratively include mannitol, sorbitol, maltitol, dextrose, sucrose, xylitol, lactose, and mixtures thereof.

The binder in the present invention is used to add cohesiveness to the composition to provide the necessary bonding to form a cohesive mass or compact upon compression. These binders are conventionally used in direct compression tablets and are described in Lieberman et al., Pharmaceutical Dosage Forms, 2 Ed., Vol. 1, pp. 209-214 (1990). Representative binders illustratively include cellulose, cellulosic derivatives, polyvinyl pyrrolidone, starch, modified starch, and mixtures thereof.

The action of chewing tablets of an inventive composition causes particulate of the tablets to adhere to pharyngeal tissue. The adhered particles deliver high local concentration dosing to those tissues relative to systemically absorbed and distributed inventive composition components. A process of pharyngeal inflammation treatment associated with infection involves the simultaneous or successive chewing of an inventive composition. Successive, as used herein, is defined as occurring sequentially with no more than a 15-minute interval between chewing events.

Typical and preferred compositions for an inventive tablet composition are summarized in Table 1.

TABLE 1
Inventive Compositions Typical and Preferred

| | Typical Amount | Preferred Amount |
|---|---|---|
| Component | Per Dose (mg) | Per Dose (mg) |
| Prostaglandin inhibitor | 50-800 | 200-500 |
| (acetylsalicylic acid) | | |
| Immunological active organic acid | 50-500 | 100-350 |
| (ascorbic acid) | | |
| Zinc cations | 0.001-0.4 | 0.001-0.05 |
| Capsaicin | 0-0.1 | 0.0001-0.01 |
| Natural Essential Oil | 0-100 | 5-20 |
| Binder | 5-400 | 50-200 |
| Compressible carbohydrate | 50-1000 | 100-500 |
| Lubricant | 0.5-20 | 2-10 |
| Masking agent | 0-50 | 5-25 |
| Flavorant | 0-500 | 50-100 |
| Color | 0-10 | 1-2 |

The present invention is further illustrated with respect to the following nonlimiting examples. These examples illustrate specific embodiments of tie present invention and are not intended to limit the scope of the appended claims.

### EXAMPLE 1

A ribbon blender is charged with 350 grams of powdered acetylsalicylic acid, 200 grams of ascorbic acid, 21 milligrams of USP grade zinc chloride, and 5 milligrams of cayenne pepper powder are mixed together for 5 minutes. 200 grams of ascorbic acid are dissolved in a minimal quantity of water at 100° C. 4 grams of starch are added to the ascorbic acid solution with the addition of necessary deionized water to wholly suspend the starch with an additional 2 minutes of agitation. An additional 16 grams of starch is added in a solution with 3 minutes of mixing and the resultant suspension passed though a screened tornado mill with the filtered solution then dried in a fluid bed dryer having an inlet temperature of 70° Celsius and an outlet temperature of 35° Celsius to obtain a moisture content of less than 2%. The dried mixture obtained from the fluid bed dryer is passed through a 20 mesh screen with an oscillator and the resultant screen powder combined with the ribbon blender mixture along with 5 grams of USP magnesium stearate. The combined material is passed through a rotary press equipped with circular shaped tablet punches to produce 1000 tablets.

### EXAMPLE 2

The procedure of Example 1 is repeated with the exception of an equimolar amount of ibuprofen replacing acetylsalicylic acid, and the inclusion of 50 grams oil or oregano and an additional 20 grams of starch. The combined material is passed through a rotary press equipped with circular shaped tablet punches to produce 1200 tablets.

### EXAMPLE 3

A group of 20 patients presenting undifferentiated throat redness and discomfort was divided into 2 groups after each patient screened negative for beta-hemolytic streptococcus infection. The first group of 10 patients was given a single chewable tablet of Example 1 while the second group was given a like dimensioned tablet placebo. Each patient was asked to chew the tablet initially with only incidental swallowing and no consumption of water for 30 minutes. Each patient was asked to rate the discomfort relief at that time with 8 out of 10 patients receiving an inventive tablet composition indicating significant discomfort relief while only 1 of the 10 patients receiving the placebo indicated the same.

### EXAMPLE 4

The process of Example 1 is repeated exclusive of acetylsalicylic acid and an equal quantity of sucrose used as replacement. The resultant tablets are administered simultaneously with a chewable 350 mg aspirin tablet to good effect consistent with Example 3 results.

### EXAMPLE 5

The process of Example 1 is repeated exclusive of acetylsalicylic acid and the cayenne pepper powder, with an equal quantity of sucrose used as replacement. The resultant tablets are administered simultaneously with a chewable 350 mg aspirin tablet to good effect consistent with Example 3 results.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A chewable composition comprising:
a first chewable prostaglandin inhibitor tablet comprising a prostaglandin inhibitor devoid of all but impurities of ascorbic acid, citric acid, and zinc cations; and
a second chewable supplement tablet comprising an immune system activating organic acid of ascorbic acid, citric acid, or a combination thereof; and
zinc cations.

2. The composition of claim 1 wherein said prostaglandin inhibitor is acetylsalicylic acid.

3. The composition of claim 1 wherein said organic acid is ascorbic acid.

4. The composition of claim 1 wherein said zinc cations are present as an inorganic zinc salt.

5. The composition of claim 2 wherein said acetylsalicylic acid is present in a range of from 200 to 500 milligrams.

6. A chewable composition comprising:
a first chewable tablet comprising a prostaglandin inhibitor and excipients compressive to a chewable prostaglandin inhibitor tablet;
a chewable dosage form comprising an immune system activating organic acid of ascorbic acid, citric acid, or a combination thereof;
zinc cations; and
excipients compressive to one or more chewable supplement tablets.

7. The composition of claim 6 wherein said prostaglandin inhibitor is acetylsalicylic acid.

8. The composition of claim 6 wherein said organic acid is ascorbic acid.

9. The composition of claim 6 wherein said zinc cations are present as an inorganic zinc salt.

10. The composition of claim 6 further comprising capsaicin.

11. The composition of claim 10 wherein said capsaicin is present as a pepper fruit powder.

12. The composition of claim 6 wherein said one or more chewable supplement tablets is one tablet.

13. The composition of claim 6 wherein said immune system activating organic acid is present in only a first chewable supplement tablet, and said zinc cations are present in only a second chewable supplement tablet.

14. A chewable composition consisting of:
between 50 and 800 milligrams of acetylsalicylic acid in a first tablet;
between 50 and 500 milligrams ascorbic acid;
between 0.001 and 0.4 milligrams zinc cations;
pharmaceutically acceptable excipients compressive to a chewable tablet form;
optionally including a natural essential oil; and
optionally including capsaicin.

15. A process for treating infective throat inflammation comprising: a subject simultaneously or successively chewing the composition of claim 6.
